(19) United States
(12) Patent Application Publication
(10) Pub. No.: US 2026/0041778 A1
(43) Pub. Date: Feb. 12, 2026

(54) SHEAR RESPONSIVE APTAMER-BASED CONJUGATE FOR TARGETED DRUG DELIVERY

(71) Applicant: Lehigh University, Bethlehem, PA (US)

(72) Inventors: Xuanhong CHENG, Bethlehem, PA (US); Xiaohui (Frank) ZHANG, Wellesley Hills, MA (US)

(73) Assignee: Lehigh University, Bethlehem, PA (US)

(21) Appl. No.: 19/291,894

(22) Filed: Aug. 6, 2025

Related U.S. Application Data
(60) Provisional application No. 63/679,798, filed on Aug. 6, 2024.

(51) Int. Cl. A61K 47/54; A61K 38/48; A61K 47/68; A61K 47/69

(52) U.S. Cl. CPC A61K 47/549; A61K 38/482; A61K 47/6849; A61K 47/6927; C12Y 304/21068

(57) ABSTRACT

The present invention relates to drug delivery systems, and methods of use therein, for the release of therapeutic payloads in response to pathological shear forces, enabling precise control over when and where biologically active agents are deployed. The drug delivery system with structures and functions responsive to shear forces comprises a microparticle, an aptamer, an antibody or antibody binding fragment thereof, and a drug. The system consists of a shear-amplifying microparticle conjugated to an aptamer that functions as a molecular transducer. Upon exposure to pathological flow conditions the drag force on the microparticle induces a conformational change in the aptamer, modulating the release or bioavailability of a drug at a therapeutically relevant location. To promote selective accumulation of the released drug, the construct is functionalized with a monoclonal antibody, which targets the therapeutically relevant location. This dual-targeted, shear-responsive system provides effective localized drug delivery.

### CROSS-REFERENCE TO RELATED APPLICATIONS

This application claims the benefit of priority from U.S. Provisional Application No. 63/679,798, filed Aug. 6, 2024, the contents of which are hereby incorporated herein by reference in their entirety.

### GOVERNMENT LICENSE RIGHTS

This invention was made with government support under 2004475 awarded by the National Science Foundation. The government has certain rights in the invention.

### BACKGROUND

Blood clot formation can obstruct or block blood vessels, potentially causing life-threatening conditions like ischemic stroke, acute myocardial infarction, and various other cardiovascular complications. Timely removal of arterial thrombi is critical for the rapid restoration of perfusion and effective management of thromboembolic diseases. In clinical practice, restoring blood flow to an occluded vessel is typically achieved either through intravenous administration of fibrinolytic agents or via interventional procedures such as percutaneous or surgical recanalization. The three primary classes of fibrinolytic agents include tissue plasminogen activator (TPA), streptokinase (SK), and urokinase (UK). Among these, TPA is favored in clinical practice due to its high specificity for fibrin and a lower risk of systemic fibrinolysis.

TPA is a serine protease that plays a central role in the fibrinolytic system by catalyzing the conversion of plasminogen to plasmin, an enzyme responsible for degrading fibrin and fibrinogen, which are the main structural components of blood clots. Endogenously secreted by endothelial cells, TPA contributes to hemostatic balance by promoting the dissolution of fibrin clots and preventing excessive thrombus formation within the vasculature. Importantly, TPA has a weak affinity for plasminogen in the absence of fibrin, with a K_{M} value of 65 μM, but its affinity increases significantly in the presence of fibrin, with a Michaelis constant, K_{M} values ranging between 0.15 and 1.5 μM. This enhanced affinity results from the assembly of TPA and plasminogen on the fibrin surface, where plasminogen binds through specific lysine-binding sites. This localization ensures that plasmin generation is spatially restricted to the clot site, allowing tightly regulated fibrinolysis. The fibrin-dependent mechanism highlights the clinical value of TPA in targeted thrombolytic therapy.

Despite the therapeutic benefits of TPA in thrombolytic therapy, its short circulation life, 2-6 minutes, necessitates large drug doses for effective thrombolysis. This requirement for excessive administration can impair normal hemostatic capabilities and lead to unwanted bleeding complications, particularly hemorrhage, thereby limiting TPA's broader applications. To address these challenges, achieving localized delivery of TPA to the site of thrombosis is essential.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description and brief description of the drawings provided below.

Aspects of the disclosure are generally directed to a drug delivery system, and methods of use therein, for the release of therapeutic payloads in response to pathological shear forces, enabling precise control over when and where biologically active agents are deployed. The system consists of a shear-amplifying microparticle conjugated to an aptamer that function as molecular transducer. Upon exposure to pathological flow conditions, the drag force on the microparticle induces a conformational change in the aptamer, modulating the release or bioavailability of the therapeutic agent at the target site. To promote selective accumulation of the released therapeutic agent at the target site, the construct is functionalized with an antibody, which targets biomolecules expressed at the target site. For example, to promote selective accumulation of released TPA at a thrombus, the construct is functionalized with a monoclonal anti-human integrin αIIbβ3 antibody, which targets integrins expressed on activated platelets enriched within forming blood clots. This dual-targeted, shear-responsive system offers a promising strategy for achieving localized drug delivery with minimal systemic complications. For example, localized TPA release at a blood clot provides localized fibrinolysis while minimizing systemic bleeding complications.

In accordance with an aspect of the invention, provided is a drug delivery system comprising a microparticle, an aptamer, an antibody or antibody binding fragment thereof, and a drug. In some embodiments the drug is a tissue plasminogen activator (TPA) or a biomolecule capable of dissolving a thrombosis. In some embodiments the tissue plasminogen activator is selected from alteplase, reteplase, and tenecteplase.

In some embodiments the microparticle is a polystyrene microparticle, a silica microparticle, a polymer microparticle, a liposomal microparticle, a gold microparticle, or another colloid microparticle. In some embodiments the microparticle has a diameter of between about 0.1 μm and about 50 μm, about 0.5 μm and about 20 μm, or about 1 μm and about 10 μm. In some embodiments the microparticle is a polystyrene bead. In some embodiments the microparticle has a diameter of about 1 μm.

In some embodiments the antibody or antibody binding fragment thereof targets a thromboembolic region. In some embodiments the antibody or antibody binding fragment thereof is a monoclonal anti-human integrin αIIbβ3 antibody. In some embodiments the aptamer is capable of binding the drug. In some embodiments the aptamer is an anti-human TPA aptamer. In some embodiments the aptamer is modified to conjugate with other biomolecules and microparticles. In some embodiments the aptamer is modified to bind the drug. In some embodiments the aptamer is modified with biofunctional groups to bind the drug and/or conjugate with other biomolecules and microparticles. In some embodiments the aptamer has been modified to include a thiol group, a biotin group, and/or another biofunctional group. In some embodiments the aptamer is modified to comprise a thiol group at a 5′ end of the aptamer and a biotin group at a 3′ end of the aptamer.

In some embodiments the aptamer is conjugated to the antibody or antibody binding fragment thereof and the microparticle. In some embodiments the drug is bound to the aptamer. In some embodiments the drug is released from the drug delivery system when in a fluid with a shear rate indicative of a thromboembolic region. In some embodiments the drug is released from the drug delivery system when in a fluid with a shear rate of 3,500 s⁻¹ or greater.

In accordance with an aspect of the invention, provided is a method of treating and/or preventing a disorder in a subject in need thereof, comprising administering to the subject in need thereof a therapeutically effective amount of the drug delivery system of any of the embodiments of the present disclosure. In some embodiments the disorder is a thromboembolic disorder selected from the group consisting of arterial cardiovascular thromboembolic disorders, venous cardiovascular thromboembolic disorders, and thromboembolic disorders in the chambers of the heart or in the peripheral circulation. In some embodiments the disorder is a thromboembolic disorder selected from unstable angina, an acute coronary syndrome, atrial fibrillation, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis.

Compared to previously reported systems, the presently disclosed drug delivery system offers improved specificity and control for localized therapy. Its dual-targeting design combines shear-responsive release with targeting antibodies, enabling precise therapeutic agent delivery at the target site while minimizing systemic exposure. The platform's modularity also allows tunable release based on flow conditions and construct design. Collectively, these attributes establish the presently disclosed drug delivery system as a mechanistically advanced and clinically relevant strategy for enhancing drug delivery while mitigating systematic complications.

### BRIEF DESCRIPTION OF THE DRAWINGS

The following detailed description of various embodiments of the invention will be better understood when read in conjunction with the appended drawings. For the purpose of illustrating the invention, certain embodiments are shown in the drawings. It should be understood, however, that the invention is not limited to the precise arrangements and instrumentalities of the embodiments shown in the drawings.

FIG. 1 The box on the left depicts the folded TPA-SMORES construct, which consists of a shear-responsive aptamer conjugated to tissue plasminogen activator (TPA) and an anti-αIIbβ3 antibody, immobilized on a polystyrene (PS) bead. Upon encountering elevated shear flow at the thrombus site, the aptamer undergoes a conformational change, resulting in the release of functional TPA. The released TPA binds to fibrin-bound plasminogen at the clot interface, catalyzing its conversion to plasmin. Plasmin then degrades the fibrin network, leading to clot dissolution and promoting fibrinolysis, ultimately restoring blood flow.

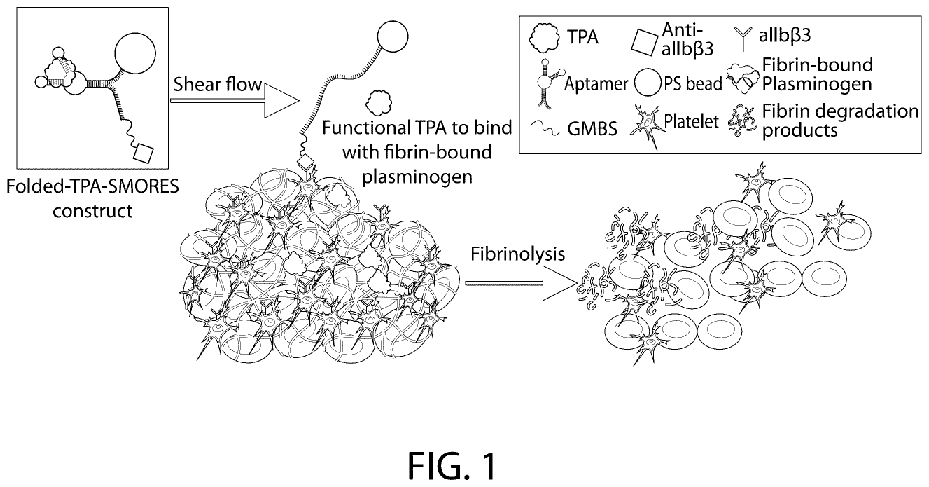

FIG. 2 (A) Representative MST binding curves showing the change in normalized fluorescence over time for a serial dilution of human TPA incubated with a constant concentration of Alexa Fluor 488-labeled anti-TPA aptamer. (B) Dose-response curve plotting normalized fluorescence against TPA concentration. A quadratic binding model was fitted to the data, yielding a dissociation constant, K_{d} of 2.03±0.78 nM, confirming high-affinity binding between the aptamer and human TPA.

FIG. 3 Fluorescence intensity of flow-through samples collected from microfluidic devices immobilized with QD-labeled TPA-SMORES constructs. Results show one distinct peak in fluorescence intensity at 50 μL/min. Data represent the average fluorescence reading from four independent microfluidic devices, with each device exposed to the full flow range in a stepwise manner. Error bars represent the standard error of the mean.

FIG. 4 COMSOL Multiphysics simulation results showing the hydrodynamic force on the bead (Fₛ) and the corresponding estimated force on the aptamer-containing tether (F_{b}) as a function of inlet flow rate. The simulation was performed using a one-micrometer bead immobilized on the microfluidic channel wall under creeping flow conditions. As the flow rate increases from 20 to 100 μL/min, the hydrodynamic force (Fₛ) acting on the bead increases accordingly. This force was then translated to the effective force on the 30.7 nm-long aptamer tether (F_{b}) using a geometric relationship that accounts for the angle between Fₛ and F_{b}.

FIG. 5 Schematic and representative images of the microfluidic clot dissolution assay used to evaluate the enzymatic activity of released TPA. (A) Schematic of the microfluidic device featuring a main channel with multiple side channels designed for clot formation (left). Images showing the loading and preparation steps (right). (B) Bar graph showing absorbance at 405 nm measured for different known concentrations of recombinant human TPA after 30 minutes of incubation, reflecting clot degradation. (C) Corresponding linear regression plot of absorbance versus TPA concentration, establishing a standard curve for quantitative assessment of TPA activity. (D) Graph representing the concentration of enzymatically active TPA released from the TPA-SMORES construct at different flow rates, based on absorbance measurements from a microfluidic clot dissolution assay and calculated using the standard calibration curve. Data represents the average of at least three independent microfluidic devices, and error bars indicate the standard error of the mean (SEM).

All drawings are not necessarily to scale. It should be understood that the various aspects are not limited to the compositions, arrangements, and instrumentality shown in the figures.

### DETAILED DESCRIPTION

The features and benefits of the invention are illustrated and described herein by reference to exemplary (“example”) embodiments. This description of exemplary embodiments is intended to be read in connection with the accompanying drawings, which are to be considered part of the entire written description. Accordingly, the disclosure expressly should not be limited to such exemplary embodiments illustrating some possible non-limiting combination of features that may exist alone or in other combinations of features. Additionally, all components and elements positively set forth in this disclosure can be negatively excluded from the claims.

All references cited herein are hereby incorporated by referenced in their entireties. In the event of a conflict in a definition in the present disclosure and that of a cited reference, the present disclosure controls. Unless defined otherwise, all technical and scientific terms used herein have the same meanings as commonly understood by one of ordinary skill in the art to which this disclosure belongs.

As used throughout, any ranges disclosed herein are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species, compounds, components, and/or elements that define or exemplify the genus is open, and it is given that other species may exist that define or exemplify the genus just as well as, or better than, any other species listed.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

The abbreviations and symbols as used herein, unless indicated otherwise, take their ordinary meaning. The symbols “hr”, “min”, “s⁻¹”, “kDa”, “mM”, “mL”, “μL”, “nm”, and “μm” refer to hour, minute, inverse second, kilodalton, millimolar, milliliter, microliter, nanometer, and micrometer, respectively.

The abbreviation “QD” means quantum dots.

The abbreviation “SMORES” refers to Single-Molecule based materials with structures and functions REsponsive to Shear.

The abbreviation “TPA” means tissue plasminogen activator, and may refer to any tissue-type plasminogen activator, recombinant tissue plasminogen activator, or other biomolecule able to dissolve thrombosis, such as alteplase, reteplase, and tenecteplase.

The term “about” when referring to a number means any number within a range of 10% of the number. For example, the phrase “about 2 μm” refers to a number between and including 1.8 μm and 2.2 μm.

The term “microparticle” refers to a particle ranging from about 0.01 μm and about 1000 μm in size.

The term “antibody or antibody binding fragment thereof” refers to one or more polypeptide chains which exhibit a strong monovalent, bivalent, or polyvalent binding to a given antigen, epitope, or epitopes.

The terms “thrombosis” and “thromboembolic” refers to formation or presence of a blood clot, clotting within a blood vessel that may cause ischemia, or infarction of tissues supplied by a blood vessel.

The term “aptamer” refers to a short oligonucleotide or RNA polynucleotide which folds into a molecular structure that bind with high affinity and specificity to target proteins, peptides, small molecules, ions, biomolecules, or cells.

The term “therapeutically effective” refers to a dosage sufficient to treat a disease, disorder, or condition for which a drug is prescribed. As used herein, the terms “treating,” “treatment,” and “treat” include (i) preventing a particular disease or disorder from occurring in a subject who may be predisposed to the disease or disorder but has not yet been diagnosed as having it; (ii) curing, treating, or inhibiting the disease or disorder, i.e., arresting its development; or (iii) ameliorating the disease or disorder by reducing or eliminating symptoms, conditions, and/or by causing regression of the disease or disorder.

Disclosed herein is a drug delivery system, and methods of use therein, for the release of therapeutic payloads in response to pathological shear forces. The systems and methods disclosed herein may advantageously be used for the localized delivery of therapeutic agents for the treatment of disorders in a subject in need thereof.

In a first embodiment of the invention, the drug delivery system comprises a microparticle, an aptamer, an antibody or antibody binding fragment thereof, and a drug.

In general, any biocompatible material well known in the art for fabrication of microparticles can be used for the microparticles of the present invention. In some embodiments the microparticle is a polystyrene microparticle, a silica microparticle, a polymer microparticle, a liposomal microparticle, a gold microparticle, or a colloid microparticle. In certain embodiments the microparticle is a polystyrene microparticle. In certain embodiments the microparticle is coated. Generally, the microparticle can be coated with any material well known in the art for coating microparticles. The coating may provide additional functionality to the microparticle, such as a point of attachment, a conjugation site, hydrophilic properties, or lipophilic properties. In certain embodiments the microparticle is a streptavidin-coated polystyrene microparticle. The microparticle may be an irregular shape or a regular shape. Thus, the microparticle can be, but is not limited to, spherical, rod, elliptical, cylindrical, disc, or any other shape.

In some embodiments the microparticle has a diameter of between about 0.1 μm and about 100 μm. In some embodiments the microparticle has a diameter of between about 0.1 μm and about 50 μm, about 0.5 μm and about 20 μm, or about 1 μm and about 10 μm. The microparticle may have a diameter of about 1 μm to about 500 μm, about 0.5 μm to about 200 μm, about 1 μm to about 200 μm, about 1 μm to about 150 μm, about 1 μm to about 100 μm, about 1 μm to about 75 μm, about 1 μm to about 50 μm, about 1 μm to about 40 μm, about 1 μm to about 30 μm, about 1 μm to about 20 μm, about 1 μm to about 5 μm, about 0.5 μm to about 50 μm, about 0.5 μm to about 40 μm, about 0.5 μm to about 30 μm, about 0.5 μm to about 10 μm, about 0.5 μm to about 5 μm. In certain embodiments the microparticle has a diameter of about 0.5 μm, about 1 μm, about 2 μm, about 3 μm, about 4 μm, about 5 μm, about 6 μm, about 7 μm, about 8 μm, about 9 μm, or about 10 μm. In certain embodiments the microparticle has a diameter of about 1 μm.

In some embodiments the drug is a thrombolytic drug, a clot-busting drug, or a fibrinolytic drug. In some embodiments the drug is a tissue plasminogen activator (TPA) or a biomolecule capable of dissolving a thrombosis. In certain embodiments the drug is alteplase, reteplase, or tenecteplase. In certain embodiments the drug is anistreplase. In certain embodiments the drug is Streptokinase (SK). In certain embodiments the drug is prourokinase, urokinase (UK), or urokinase-type plasminogen activator (uPA). The drug may also be an antiplatelet drug, such as acetylsalicylic acid (ASA), cangrelor, cilostazol, clopidogrel, dipyridamole, prasugrel, ticlopidine, ticagrelor, abciximab, eptifibatide, tirofiban, or vorapaxar. The drug may also be an anticoagulant, such as heparin, dalteparin, enoxaparin, tinzaparin, heparinoid, warfarin, apixaban, edoxaban, fondaparinux, rivaroxaban, argatroban, bivalirudin, dabigatran, desirudin, or lepirudin.

The antibodies or antibody binding fragments thereof of the drug delivery system of the present invention typically retain the antigen binding capability of their native, unconjugated counterparts. The antibody or antibody binding fragment thereof may be a monoclonal antibody, a human chimeric antibody, a humanized antibody, a human antibody, a recombinant human antibody, an anti-human antibody, or an integrin antibody. In certain embodiments the antibody or antibody binding fragment thereof is a monoclonal anti-human integrin antibody. In certain embodiments the antibody or antibody binding fragment thereof targets a thromboembolic region. In certain embodiments the antibody or antibody binding fragment thereof is a monoclonal anti-human integrin αIIbβ3 antibody.

In some embodiments the aptamer is capable of binding the drug of the drug delivery system. In certain embodiments the aptamer is an anti-human TPA aptamer. In certain embodiments the aptamer is an anti-human PLAT aptamer.

The aptamer may be between about 10 and about 200 nucleotides in length. The aptamer may be between about 20 and about 100 nucleotides in length. The aptamer may be between about 30 and about 80 nucleotides in length. The aptamer may be between about 40 and about 60 nucleotides in length. The aptamer may be between about 50 and about 55 nucleotides in length. The aptamer may be between about 45, about 46, about 47, about 48, about 49, about 50, about 51, about 52, about 53, about 54, about 55, about 56, about 57, about 58, about 59, or about 60 nucleotides in length. In certain embodiments the aptamer is about 52 nucleotides in length.

The aptamer of the present invention may be modified by any means well known in the art. In certain embodiments the aptamer is modified to bind the drug of the drug delivery system. In certain embodiments the aptamer is capable of binding the drug of the drug delivery system without modification. In certain embodiments the aptamer is modified to increase the binding affinity of the aptamer for the drug.

In some embodiments, the aptamer comprises at least one chemical modification. In some embodiments, the aptamer comprises at least two chemical modifications. In some embodiments, the chemical modification is selected from a chemical substitution of the nucleic acid at a sugar position, a chemical substitution at a phosphate position, and/or a chemical substitution at a base position. In some embodiments, the chemical modification is selected from incorporation of a modified nucleotide; 3′ capping; 5′ capping; conjugation to a non-immunogenic compound; conjugation to a lipophilic compound; and incorporation of phosphorothioate into the phosphate backbone. In certain embodiments the aptamer may be modified to comprise a thiol group, a biotin group, a hydroxyl group, an amine group, an amide group, an aldehyde group, a nitrile group, a nitro group, a carboxylic acid, and/or another biofunctional group. In certain embodiments the aptamer is modified to comprise a thiol group and/or a biotin group. In certain embodiments the aptamer is modified at a 3′ end of the aptamer. In certain embodiments the aptamer is modified at a 5′ end of the aptamer. In certain embodiments the aptamer is modified at a 5′ end of the aptamer and at a 3′ end of the aptamer. In certain embodiments the aptamer is modified to comprise a thiol group at a 5′ end of the aptamer and a biotin group at a 3′ end of the aptamer.

In some embodiments the aptamer is conjugated to an antibody or antibody binding fragment thereof. In some embodiments the aptamer is conjugated to a microparticle. In some embodiments the aptamer is conjugated to an antibody or antibody binding fragment thereof and a microparticle.

In some embodiments the drug is bound to the aptamer. In some embodiments the drug is released from the drug delivery system when in a fluid with a shear rate indicative of a disorder. In some embodiments the drug is released from the drug delivery system when in a fluid with a shear rate indicative of a thromboembolic region.

Generally, aptamers bind specific target proteins, peptides, small molecules, ions, biomolecules, or cells by folding into a three-dimensional conformation based on the chemical structure of the aptamer. The folded aptamer creates a binding pocket which binds specific targets with high specificity. The present invention uses shear force to unfold the binding pocket and thereby release the target bound therein. In exemplary embodiments, an antibody or antibody binding fragment thereof conjugated to the aptamer will bind to a target at the location of a disorder or a thromboembolic region. The bound antibody or antibody binding fragment thereof will act as an anchor on the aptamer, which under shear flow forces will be pulled away from the antibody or antibody binding fragment thereof unfolding the three-dimensional binding site and releasing the drug bound therein. In exemplary embodiments, the aptamer is also conjugated to a microparticle which increases the surface area of the drug delivery system and therefore increases the force exerted on the anchored aptamer by the shear flow force. The aptamer can also be shortened or lengthened. By optimizing the microparticle size and the aptamer or tether length the system can be customized to have shear responsiveness to match a broad range of pathological shear rates observed in vascular disorder.

In healthy vasculature, blood shear rates vary depending on the vessel type. Physiological shear rates of in vivo circulating blood in healthy arteries (300-800 s⁻¹), veins (15-200 s⁻¹), and microvessels (450-1800 s⁻¹), supporting normal endothelial function and vascular homeostasis. Physiological conditions, such as exercise, can transiently elevate arterial shear rates up to approximately 1000 s⁻¹ due to increased blood flow, as wall shear rate (WSR) scales with flow rate and inversely with vessel diameter. However, under pathological conditions, such as atherosclerotic stenosis, shear rates can rise dramatically. Due to the narrowing of the vessel lumen, shear rates may exceed 5000 s⁻¹ and, in severe cases, reach levels greater than 200,000 s⁻¹.

When the drug is bound to the aptamer the drug may be released from the drug delivery system when in a fluid with a shear rate above a physiological level. When the drug is bound to the aptamer the drug may be released from the drug delivery system when in a fluid with a flow rate of between about 20 μL/min and about 120 μL/min, about 40 L/min and about 60 μL/min, and about 90 μL/min and about 110 μL/min. When the drug is bound to the aptamer the drug may be released from the drug delivery system when in a fluid with a flow rate of about 50 μL/min. When the drug is bound to the aptamer the drug may be released from the drug delivery system when in a fluid with a flow rate of about 50 L/min or greater. When the drug is bound to the aptamer the drug may be released from the drug delivery system when in a fluid with a shear rate of between about 1405 s⁻¹ and about 7026 s⁻¹, about 2810 s⁻¹ and about 4215 s⁻¹, and about 6323 s⁻¹ and about 7026 s⁻¹. When the drug is bound to the aptamer the drug may be released from the drug delivery system when in a fluid with a shear rate of about 3513 s⁻¹. In some embodiments the drug is released from the drug delivery system when in a fluid with a shear rate of about 3,500 s⁻¹ or greater. In some embodiments the drug is released from the drug delivery system when in a fluid with a shear rate of about 2,000 s⁻¹ or greater. In some embodiments the drug is released from the drug delivery system when in a fluid with a shear rate of about 1,800 s⁻¹ or greater. In some embodiments the drug is released from the drug delivery system when in a fluid with a shear rate of about 1,500 s⁻¹ or greater. In some embodiments the fluid is a bodily fluid. In some embodiments the fluid is blood, blood plasma, water, plasma expanders, saline, colloidal solutions, human serum albumin, or a combination of any of the foregoing.

The drug delivery system of the present application may be used in a method of treating and/or preventing a disorder. The drug delivery system of the present application may be used in a method of treating and/or preventing a disorder in a subject in need thereof, comprising administering to the subject in need thereof a therapeutically effective amount of the drug delivery system.

In one aspect, the drug delivery system of the present application may be used in a method of treating and/or preventing a thromboembolic disorder in a subject in need thereof, comprising administering to the subject in need thereof a therapeutically effective amount of the drug delivery system. In some embodiment, the present disclosure relates to the use of the drug delivery system in the manufacture of a medicament for treating a thromboembolic disorder. In some embodiment, the present disclosure relates to the drug delivery system for use in the treatment of a thromboembolic disorder. In some embodiments the thromboembolic disorder is selected from the group consisting of arterial cardiovascular thromboembolic disorders, venous cardiovascular thromboembolic disorders, and thromboembolic disorders in the chambers of the heart or in the peripheral circulation. In some embodiments the thromboembolic disorder is selected from unstable angina, an acute coronary syndrome, atrial fibrillation, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. In certain embodiments the thromboembolic disorder is stroke. In certain embodiments the thromboembolic disorder is myocardial infarction.

In some embodiments the administrating of the drug delivery system to the subject in need thereof is administered by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof. In some embodiments, the drug delivery system is administered as a dose of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mg/kg. In some embodiments, the drug delivery system is administered as a dose of about 1 mg to about 100 mg, about 10 mg to about 90 mg, about 20 mg to about 80 mg, about 30 mg to about 40 mg, about 40 mg to about 50 mg, about 50 mg to about 60 mg, about 60 mg to about 70 mg, or about 80 mg to about 90 mg. In some embodiments, the drug delivery system is administered as a dose of about 1, 2, 3, 4, 5, 6, 7, 8, 9, 10, 15, 20, 25, 30, 35, 40, 45, 50, 55, 60, 65, 70, 75, 80, 85, or 90 mg.

The presently disclosed drug delivery system is a highly versatile and advanced system for controlled, site-specific drug delivery, offering significant improvements over existing shear-sensitive delivery strategies. Compared to previously reported systems, the presently disclosed SMORES system offers improved specificity and control for localized therapy. Its dual-targeting design combines shear-responsive release with site specific antibodies, enabling precise delivery at the disorder location while minimizing systemic exposure. The system's modularity also allows tunable release based on flow conditions and construct design. Collectively, these attributes establish SMORES as a mechanistically advanced and clinically relevant strategy for enhancing therapeutic efficacy while mitigating systematic complications.

### EXAMPLES

Abbreviations

| | |
|---|---|
| BSA | Bovine Serum Albumin |
| EDTA | Ethylenediaminetetraacetic Acid |
| GMBS | N-γ-Maleimidobutyryl-Oxysuccinimide Ester |
| MST | Microscale Thermophoresis |
| MWCO | Molecular Weight Cut-Off |
| PBS | Phosphate-Buffered Saline |
| QD | Quantum Dot |
| TIRF | Total Internal Reflection Fluorescence |
| v/v | volume per volume |
| w/v | weight per volume |

### Example 1

A Single-Molecule based materials with structures and functions REsponsive to Shear (SMORES) drug delivery system was constructed with an anti-human PLAT aptamer comprising a thiol group at a 5′ end of the aptamer and a biotin group at a 3′ end of the aptamer. The SMORES material was constructed by first conjugating the 5′ thiolated end of the anti-PLAT aptamer molecules to a Human Integrin αIIbβ3 Antibody using N-γ-maleimidobutyryl-oxysuccinimide ester (GMBS) crosslinker. The antibody was mixed with GMBS at a 1:50 molar ratio (antibody to crosslinker) in 1×PBS supplemented with 1 mM EDTA and incubated at room temperature for 30 minutes. This allowed the oxysuccinimide ester end of the GMBS crosslinker to react with the primary amines on the antibody, facilitating crosslinking. Afterward, the reaction mixture was washed twice using Zeba Spin Desalting Columns (7 kDa MWCO) to remove excess GMBS. Immediately after washing, the antibody-GMBS conjugate was mixed with the reduced aptamer at a 1:5 molar ratio (antibody to aptamer), facilitating conjugation through the reaction between the maleimide end of the GMBS crosslinker and the 5′ thiol group on the aptamer. The aptamer-antibody conjugate was then washed twice using a 30 kDa MWCO Pierce concentrator to remove any unbound aptamer before proceeding to the next conjugation reaction. Aptamer-antibody conjugation results were validated using gel electrophoresis.

The aptamer-antibody conjugate was then reacted with 1 μM polystyrene (PS) streptavidin-coated microspheres through the interaction between the 3′ biotinylated end of the aptamer and the streptavidin on the surface of the microspheres. 50 μL of the stock beads were suspended in a microcentrifuge tube and centrifuged at 10,000×g for 3 minutes to pellet them. The supernatant was discarded, and the beads were washed three times with PBS supplemented with 1% BSA followed by centrifugation. After the final wash, the antibody-aptamer conjugate was reacted with the beads at a 1:25 mass ratio of 3′ biotin-containing conjugate to beads. The reaction was incubated for 1 hour at room temperature with gentle shaking. Following incubation, the beads conjugated to the aptamer-antibody were washed three times by centrifugation (10,000×g for 3 minutes in PBS with 1% BSA) to remove any unbound aptamer-antibody conjugate. The bead-aptamer-antibody conjugation results were validated using microfluid devices functionalized with recombinant human αIIbβ3.

### Example 2

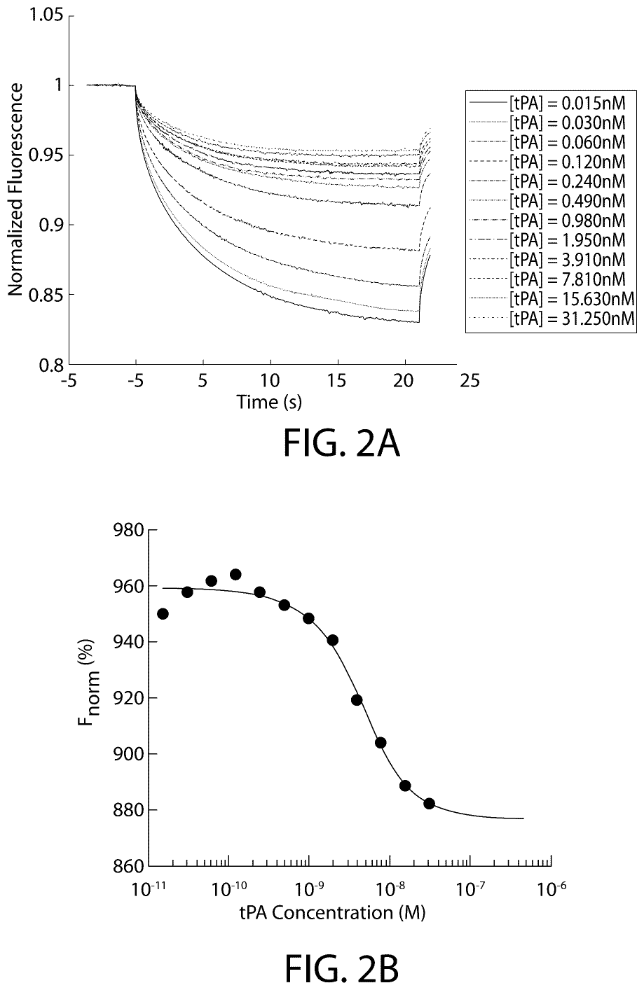

To confirm the high binding affinity between the aptamer and TPA, microscale thermophoresis (MST) was performed. As shown in FIG. 2A, normalized fluorescence traces were obtained across a serial dilution of TPA incubated with a constant concentration of fluorescently labeled aptamer. In MST, binding alters the thermophoretic mobility of the aptamer, resulting in concentration-dependent shifts in fluorescence that indicate complex formation. Equilibrium fluorescence values were plotted against TPA concentration to generate a dose-response curve, FIG. 2B, which was fitted using a quadratic binding model. This analysis yielded a dissociation constant (K_{d}) of 2.03±0.78 nM, confirming high-affinity binding between the aptamer and TPA.

### Example 3

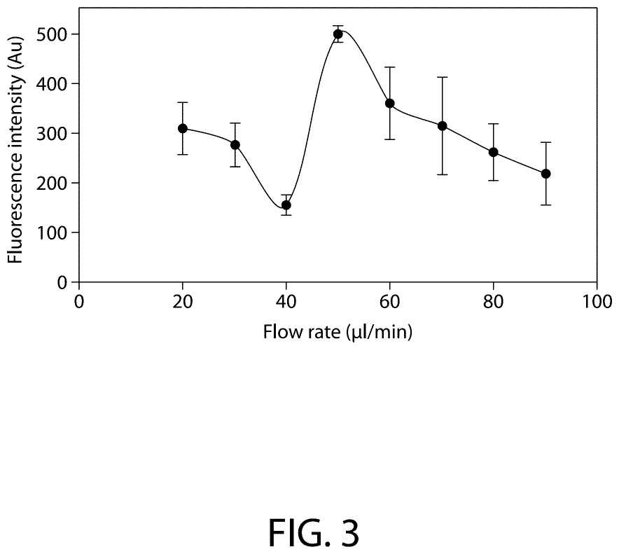

To investigate the flow-dependent modulation of the SMORES material functionality, a syringe pump was used to apply varying flow rates (10 to 100 μL/min) to a microfluidic channel functionalized with the SMORES-TPA complex. The microfluidic channel measured 50 μm in height, 8 mm in length, and 1.0 mm in width. To evaluate the aptamer's ability to unfold and release its ligand, flow-through samples containing QD-TPA were collected at each flow rate, and fluorescence intensity was measured (excitation: 480 nm; emission: 600 nm). The flow buffer used in these experiments consisted of 1% fatty acid-free BSA in PBS. Fluorescence from a negative control containing only BSA was subtracted from all experimental readings to account for background signal. This approach enabled quantification of TPA release as a function of flow rate, allowing identification of the threshold at which the TPA aptamer undergoes conformational changes and releases its target. The results are shown in FIG. 3.

The flow-through samples collected from microfluidic channels functionalized with TPA-SMORES exhibited a non-linear release profile. A pronounced peak in fluorescence intensity was observed at 50 μL/min, suggesting this flow rate represents a critical shear threshold at which the aptamer undergoes a conformational change, resulting in specific TPA release.

### Example 4

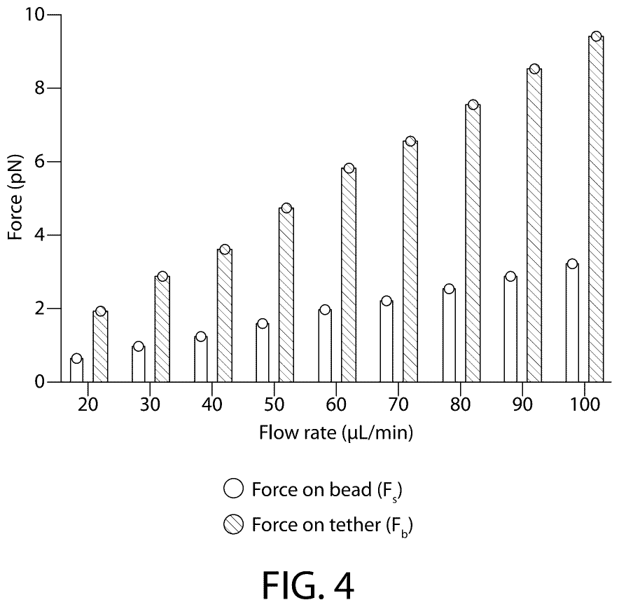

To relate the observed flow-dependent behavior of the TPA-SMORES construct to molecular-scale forces, a COMSOL Multiphysics model was developed to simulate the hydrodynamic forces acting on a microsphere tethered to the channel wall. The microfluidic channel geometry used in experiments was replicated in the simulation, and a one μm diameter bead was positioned on the wall. The simulation employed a single-phase creeping flow module under steady-state conditions, with water as the working fluid and no-slip boundary conditions applied to all surfaces. Inlet flow rates ranged from 20 to 100 μL/min, matching the experimental conditions. The simulation produced force values on the bead, Fₛ, incorporating both normal and shear stress components. To estimate the force transmitted to the 30.7-nm-long tether, comprising a 52-base aptamer and a GMBS crosslinker, F_{b}, a geometrical relationship was used, accounting for the angle, θ, between Fₛ and F_{b}. This approach incorporates the bead radius and tether length to determine the effective pulling force on the tether. The COMSOL simulation results showed that the hydrodynamic force on the bead (Fₛ) and the corresponding force transmitted to the aptamer-containing tether (F_{b}) increased progressively with the applied flow rate. At an applied flow rate of 50 μL/min, the bead experienced a force (Fₛ) of 1.62 pN, corresponding to a tether force (F_{b}) of 4.76 pN, with a maximum shear rate of 3512.9 s⁻¹ on the bead surface. The corresponding bead forces Fₛ, tether forces F_{b}, and shear rates are summarized in FIG. 4 and Table 1.

TABLE 1
Simulated hydrodynamic force on beads under different flow rates
and the corresponding pulling force on the aptamer tether.

| Flow rate | Force on bead | Force on 31.2 nm | Maximum shear rate on |
|---|---|---|---|
| (μL/min) | Fₛ (pN) | Tether F_{b} (pN) | the bead (1/s) |
| 20 | 0.67 | 1.97 | 1405.2 |
| 30 | 0.99 | 2.91 | 2107.8 |
| 40 | 1.24 | 3.65 | 2810.4 |
| 50 | 1.62 | 4.76 | 3512.9 |
| 60 | 1.99 | 5.85 | 4215.5 |
| 70 | 2.25 | 6.62 | 4918.1 |
| 80 | 2.58 | 7.59 | 5620.7 |
| 90 | 2.91 | 8.56 | 6323.3 |
| 100 | 3.22 | 9.47 | 7025.9 |

### Example 5

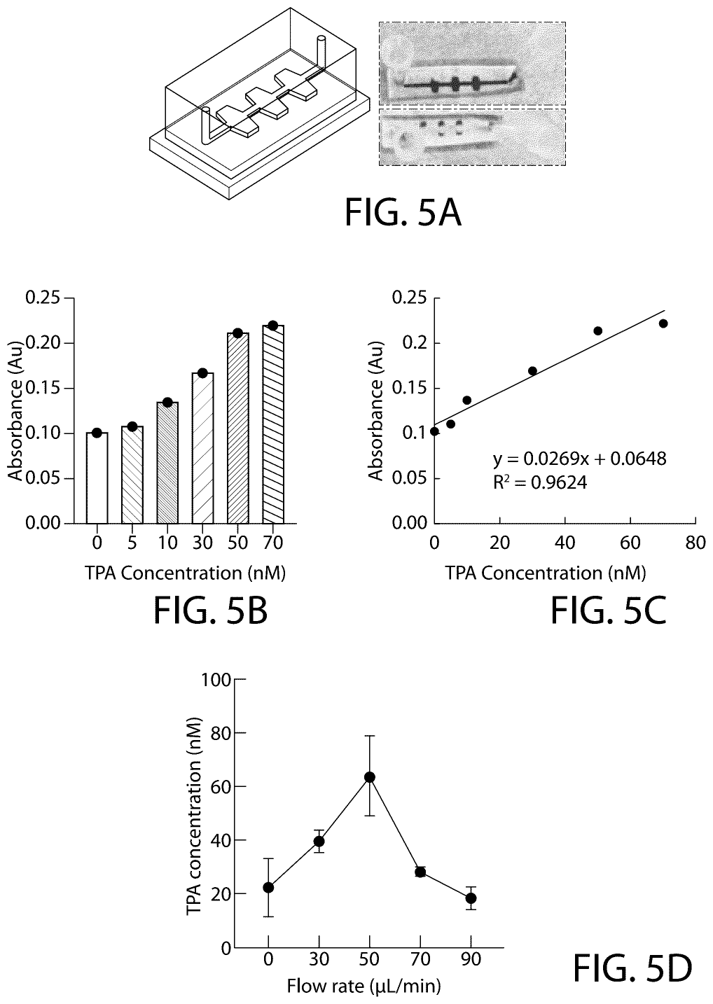

To assess the enzymatic activity of the released TPA from the TPA-SMORES complex, a functionality assay was developed to evaluate clot dissolution as a function of TPA concentration. The microfluidic device and channel layout used in this assay are illustrated in FIG. 5A. The channels were primed with PBS before use. Whole blood was mixed with 2.45% (w/v) calcium chloride at a 9:1 (v/v) ratio to initiate clotting. The mixture was introduced into the devices to coat both the main and side channels. Air was then flushed through the main channel to remove blood, leaving comparable amounts of blood in the side channels. Blood in the side channels was incubated for 40 minutes at room temperature to allow clot formation. Afterward, the main channels were washed with PBS to remove unbound components. For standard curve generation, known concentrations of recombinant human TPA (20, 40, 60, and 80 nM) were introduced into the main channel and incubated for 30 minutes on a rocker to allow diffusion into the side clots. Following incubation, devices were connected to a syringe pump, and flow-through was collected at 100 μL/min for 1 minute. Absorbance of the eluate was measured at 405 nm, corresponding to the absorbance of hemaglobin. These absorbance values were plotted against known TPA concentrations to generate a calibration curve correlating absorbance with TPA concentration.

As shown in FIG. 5B, a concentration-dependent increase in absorbance was observed, indicating progressive clot dissolution with higher TPA concentrations. The corresponding linear regression analysis, FIG. 5C, demonstrated a strong linear relationship between absorbance and TPA concentration. This standard curve provides a reliable framework for quantifying unknown TPA concentrations based on clot degradation.

Application of this assay to flow-through samples collected from the TPA-SMORES complex, followed by conversion of absorbance values into TPA concentrations using the calibration curve, revealed a flow rate-dependent release profile. As shown in FIG. 5D, a notable peak in TPA concentration was observed at 50 μL/min compared to both lower and higher flow rates, indicating that this flow condition generates sufficient shear force to unfold the aptamer and release the majority of bound TPA.

Based on the shear rate values obtained in this study, the major release of TPA from the SMORES constructs occurs at a flow rate peak of 50 μL/min, corresponding to a maximum shear rate of 3512.9 s⁻¹. This ensures that TPA release is confined to pathological flow conditions typically associated with thrombus formation, where shear rates exceed physiologic levels.

While the foregoing description and drawings represent some example drug delivery systems, it will be understood that various additions, modifications, and substitutions may be made therein without departing from the spirit and scope and range of equivalents of the accompanying claims. In particular, it will be clear to those skilled in the art that the present invention may be embodied in other forms, structures, arrangements, proportions, sizes, and with other elements, materials, and components, without departing from the spirit or essential characteristics thereof.

In addition, numerous variations in the methods/processes described herein may be made. One skilled in the art will further appreciate that the invention may be used with many modifications of structure, arrangement, proportions, sizes, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being defined by the appended claims and equivalents thereof, and not limited to the foregoing description or embodiments. Rather, the appended claims should be construed broadly, to include other variants and embodiments of the invention, which may be made by those skilled in the art without departing from the scope and range of equivalents of the invention.

What is claimed is:

1. A drug delivery system comprising a microparticle, an aptamer, an antibody or antibody binding fragment thereof, and a drug.

2. The drug delivery system according to claim 1, wherein the microparticle is a polystyrene microparticle, a silica microparticle, a polymer microparticle, a liposomal microparticle, a gold microparticle, or another colloid microparticle.

3. The drug delivery system according to claim 1, wherein the microparticle has a diameter of between about 0.1 μm and about 50 μm, about 0.5 μm and about 20 μm, or about 1 μm and about 10 μm.

4. The drug delivery system according to claim 1, wherein the drug is a tissue plasminogen activator (TPA) or a biomolecule capable of dissolving a thrombosis.

5. The drug delivery system according to claim 4, wherein the tissue plasminogen activator (TPA) is selected from alteplase, reteplase, and tenecteplase.

6. The drug delivery system according to claim 1, wherein the antibody or antibody binding fragment thereof targets a thromboembolic region.

7. The drug delivery system according to claim 1, wherein the antibody or antibody binding fragment thereof is a monoclonal anti-human integrin αIIbβ3 antibody.

8. The drug delivery system according to claim 1, wherein the aptamer is capable of binding the drug.

9. The drug delivery system according to claim 1, wherein the aptamer is an anti-human TPA aptamer.

10. The drug delivery system according to claim 1, wherein the aptamer is modified to bind the drug.

11. The drug delivery system according to claim 10, wherein the aptamer is modified to comprise a thiol group, a biotin group, and/or another biofunctional group.

12. The drug delivery system according to claim 10, wherein the aptamer is modified to comprise a thiol group at a 5′ end of the aptamer and a biotin group at a 3′ end of the aptamer.

13. The drug delivery system according to claim 1, wherein the aptamer is conjugated to the antibody and the microparticle.

14. The drug delivery system according to claim 13, wherein the drug is bound to the aptamer.

15. The drug delivery system according to claim 14, wherein the drug is released from the drug delivery system when in a fluid with a shear rate indicative of a thromboembolic region.

16. The drug delivery system according to claim 14, wherein the drug is released from the drug delivery system when in a fluid with a shear rate of 3,500 s⁻¹ or greater.

17. A method of treating and/or preventing a disorder in a subject in need thereof, comprising administering to the subject in need thereof a therapeutically effective amount of the drug delivery system according to claim 1.

18. The method according to claim 17, wherein the disorder is a thromboembolic disorder selected from the group consisting of arterial cardiovascular thromboembolic disorders, venous cardiovascular thromboembolic disorders, and thromboembolic disorders in the chambers of the heart or in the peripheral circulation.

19. The method according to claim 17, wherein the disorder is a thromboembolic disorder selected from unstable angina, an acute coronary syndrome, atrial fibrillation, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis.
